(19) United States
(12) Patent Application Publication
(10) Pub. No.: US 2024/0325769 A1
(43) Pub. Date: Oct. 3, 2024

(54) METHODS OF TREATING SPINAL CORD INJURIES WITH ALTERNATING ELECTRIC FIELDS

(71) Applicant: NOVOCURE GMBH, Root D4 (CH)

(72) Inventor: LILACH AVIGDOR, Haifa (IL)

(21) Appl. No.: 18/621,809

(22) Filed: Mar. 29, 2024

Related U.S. Application Data
(60) Provisional application No. 63/493,383, filed on Mar. 31, 2023.

(51) Int. Cl. A61N 1/40

(52) U.S. Cl. CPC A61N 1/40

(57) ABSTRACT

Disclosed are methods of treating a subject having a spinal cord injury comprising applying an alternating electric field, at a frequency for a period of time, to a target site of the subject. Disclosed herein are methods of increasing N-cadherin expression in a subject having spinal cord injury comprising applying alternating electric fields, at a frequency for a period of time, to a target site of the subject, thereby increasing N-cadherin expression at the target site in the subject. Disclosed are methods of increasing neuronal connection, axon regeneration, neuronal protection, and/or neuronal differentiation in a subject having a spinal cord injury comprising applying alternating electric fields, at a frequency for a period of time, to a target site of the subject.

### CROSS-REFERENCE TO RELATED APPLICATIONS

This application claims the benefit of U.S. Provisional Patent Application No. 63/493,383, filed Mar. 31, 2023, which is incorporated by reference herein in its entirety.

### BACKGROUND

Spinal cord injury (SCI) often causes patients to lose the sensory and motor function below the injury segment. Worldwide, the annual incidence of SCI is approximately 40 people per million. Due to the lack of effective therapeutic method, SCI often leads to lifelong disability. In addition, the cost of treating and caring for the patient over their lifetime brings a huge economic burden to their families and the society. Following SCI, cells expressing Nestin, a marker for neural stem/progenitor cells (NSPCs), become activated in spared neural tissue, thus representing a source of cells for replenishing lost neurons. However, because of the lack of proper scaffold, such as radial glial (RG) fibers and blood vessels in the injury site after complete spinal cord transection, the migration of these Nestin+ cells to the injury site is regionally restricted. A study by Li et al. has shown that on the fifth day following complete spinal cord transection in rats, the number of Nestin+ cells in spared spinal segments adjacent to the lesion area peaked; however, only a small number of Nestin+ cells spontaneously migrated to the lesion center. Furthermore, on the seventh day after SCI, numbers of Nestin+ cells in spared spinal segments began to decrease, and NSPCs continued to experience difficulty migrating to the lesion center. With the destruction of the blood brain barrier, blood-borne fibrocytes and immune cells infiltrate into the injured area together with proliferating pericytes and fibroblasts to replace the damaged neural cells. As the inflammatory response subsides and gradual deposition of chondroitin sulfate proteoglycans (CSPGs) occurs, the non-neural lesion core (stromal scar) matures, which makes the migration of NSPCs more difficult.

### BRIEF SUMMARY

Disclosed are methods of treating a subject having a spinal cord injury comprising applying an alternating electric field, at a frequency for a period of time, to a target site of the subject.

Disclosed herein are methods of increasing N-cadherin expression in a subject having a spinal cord injury comprising applying alternating electric fields, at a frequency for a period of time, to a target site of the subject, thereby increasing N-cadherin expression at the target site in the subject.

Disclosed are methods of promoting migration of neural stem progenitor cells (NSPCs) to a spinal cord injury in a subject comprising applying alternating electric fields, at a frequency for a period of time, to a target site of the subject, wherein the target site is at the site of the spinal cord injury, thereby promoting migration of NSPCs to the target site.

Disclosed are methods of increasing neuronal connection in a subject having a spinal cord injury comprising applying alternating electric fields, at a frequency for a period of time, to a target site of the subject, thereby increasing neuronal connection at the target site in the subject.

Disclosed are methods of increasing axon regeneration in a subject having a spinal cord injury comprising applying alternating electric fields, at a frequency for a period of time, to a target site of the subject, thereby increasing axon regeneration at the target site in the subject.

Disclosed are methods of increasing neuronal protection in a subject having a spinal cord injury comprising applying alternating electric fields, at a frequency for a period of time, to a target site of the subject, thereby increasing neuronal protection at the target site in the subject.

Disclosed are methods of increasing neuronal differentiation in a subject having a spinal cord injury comprising applying alternating electric fields, at a frequency for a period of time, to a target site of the subject, thereby increasing neuronal differentiation at the target site in the subject.

Additional advantages of the disclosed methods and compositions will be set forth in part in the description which follows, and in part will be understood from the description, or may be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows that N-cadherin expression is increased in ECM secreting cells (fibroblasts) following TTFields application.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a target site” includes a plurality of such target sites, reference to “the spinal cord injury” is a reference to one or more spinal cord injuries and equivalents thereof known to those skilled in the art, and so forth.

As used herein, a “target site” is a specific site or location within or present on a subject or patient. For example, a “target site” can refer to, but is not limited to a site of a spinal cord injury. For example, the phrase “target cell” can be used to refer to target site, wherein the target site is a cell. In some aspects, a “target cell” can be a neuron. In some aspects, a cell or population of cells that can be a target site or a target cell include, but are not limited to, a neuronal cell or stem cell. In some aspects, a “target site” can be a neuronal target site, wherein a neuronal target site is a site or location in a subject comprising neuronal cells. In some aspects, a target site can refer to a site or location within or present on a subject or patient that comprises a spinal cord injury. Additionally, a target site can refer to a site or location adjacent to a spinal cord injury. In some aspects, a target site can be a site within a subject that comprises one or more sites in the subject that comprise a therapeutic or therapeutic device intended to treat the subject. For example, in some aspects, a target site can comprise an implant such as a linearly ordered collagen scaffold.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as the spinal cord). In some aspects, the alternating electrical field can be in a single direction or multiple directional. In some aspects, alternating electric fields can be delivered through two pairs of transducer arrays that generate perpendicular fields within the target site. For example, for the Optune® system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

In-vivo and in-vitro studies show that the efficacy of an alternating electric field increases as the intensity of the electrical field increases. Therefore, optimizing array placement on a subject to increase the intensity in the target site or target cell is standard practice for the Optune® system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with a spinal cord injury.

By “treat” is meant to administer or apply a therapeutic, such as alternating electric fields and a vector, to a subject, such as a human or other mammal (for example, an animal model), that has a spinal cord injury or has an increased susceptibility for developing a spinal cord injury, in order to prevent or delay a worsening of the effects of the injury, or to partially or fully reverse the effects of the injury. For example, treating a subject having a spinal cord injury can comprise applying an alternating electric field and/or delivering a therapeutic to a cell or other target site in the subject.

By “prevent” is meant to minimize or decrease the chance that a subject develops a spinal cord injury or a symptom associated therewith.

As used herein, the terms “administering” and “administration” refer to any method of providing a therapeutic to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to help with spinal cord injuries. In further various aspects, a preparation can be administered prophylactically; that is, administered for prevention of spinal cord injury damage. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present methods and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 10 MHz. The frequency of the alternating electric fields can also be, but is not limited to, between 50 kHz and 1 MHz, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 10 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site. When applying alternating electric fields to a cell, this can often refer to applying alternating electric fields to a subject comprising a spinal cord injury. Thus, applying alternating electric fields to a target site of a subject results in applying alternating electric fields to a cell and/or a spinal cord injury.

### C. Methods of Treating

Disclosed are methods of treating a subject having a spinal cord injury comprising applying an alternating electric field, at a frequency for a period of time, to a target site of the subject. In some aspects, the target site is the site of the spinal cord injury.

In some aspects, application of the alternating electric field can also: increase N-cadherin expression at the target site, increase axon regeneration, neuronal protection or neuronal differentiation at the target site, and/or increase neuronal connections at the target site, thereby treating the spinal cord injury in the subject. In some aspects, N-cadherin regulates mechanical adhesion between neural stem/progenitor cells (NSPCs), and also drives NSPCs migration and promotes NSPCs differentiation, therefore an increase in N-cadherin can have therapeutic effects such as treating a spinal cord injury.

In some aspects, the spinal cord injury occurred at least 1, 2, 3, 4, 5, 6, or 7 days before applying the alternating electric fields. In some aspects, the spinal cord injury occurred at least 1, 2, 3, or 4 weeks before applying the alternating electric fields. In some aspects, the spinal cord injury occurred at least 1, 2, 3, 4, 5, 6, 7, 8, 9, 10, 11, 12, 13, 14, 15, 16, 17, 18, 19, 20, 21, 22, 23 or 24 hours before applying the alternating electric fields. In some aspects, the spinal cord injury occurred less than 1 hour before applying the alternating electric fields.

In some aspects, the subject having a spinal cord injury does not have cancer, has not been diagnosed with cancer, and/or is not being treated for cancer. Thus, in some aspects, alternating electric fields are applied to a subject that does not have cancer and/or has not yet been diagnosed with cancer. In some aspects, the target site does not comprise cancer cells and/or a tumor(s).

In some aspects, the methods disclosed herein can further comprise administering physiotherapy, muscle vibration, steroid treatment, respiratory management, and/or pain management.

In some aspects, the methods can further comprise the step of implanting a collagen scaffold to the site of the spinal cord injury before or after applying an alternating electric field, at a frequency for a period of time to the subject. In some aspects, the collagen scaffold is a linear ordered collagen scaffold. In some aspects, the collagen scaffold can promote the migration and neuronal differentiation of neural stem/progenitor cells to the spinal cord injury to aide in repair. Thus, the application of a collagen scaffold with the alternating electric fields can increase N-cadherin and can be an effective treatment for a spinal cord injury, can increase N-cadherin expression at the target site, can increase axon regeneration, can increase neuronal protection or neuronal differentiation at the target site, and/or can increase neuronal connections at the target site or at or around the collagen scaffold Because a collagen scaffold can be implanted at the site of a spinal cord injury, in some aspects, the target site for applying alternating electric fields can comprise an implanted collagen scaffold. In some aspects, the disclosed methods can further comprise applying an alternating electric field, at a frequency for a period of time to the site of the spinal cord injury after implanting a linear ordered collagen scaffold to the site of the spinal cord injury.

In some aspects, the methods can further comprise the step of introducing a biomaterial scaffold. In some aspects, introducing the biomaterial scaffold comprising implanting the biomaterial scaffold. In some aspects, biomaterial scaffolds can include, but are not limited to, hydrogels, biodegradable scaffolds, micro/nanofibers as instructive biomaterials, and drug-delivering biomaterials. For example, a biomaterial scaffold can be one or more of those scaffolds described in Suzuki et al. Current Concepts of Biomaterial Scaffolds and Regenerative Therapy for Spinal Cord Injury. Int. J. Mol. Sci. 2023, 24 (3), 2528, incorporated by reference in its entirety herein.

In some aspects, the methods further comprise ceasing application of the alternating electrical field once spinal cord connectivity is restored.

In some aspects, the alternating electric fields can have any of the frequencies or field strengths described herein. In some aspects, the frequency of the alternating electric field is between 100 kHz and 10 MHz. For example, the frequency of the alternating electric field may be between 50 kHz and 1 MHz or between 100 kHz and 500 kHz. In some aspects, the frequency of the alternating electric field is 100-300 kHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

Disclosed are compositions comprising a biomaterial scaffold for use in a method of treating a subject having a spinal cord injury comprising introducing the composition to a target site of a subject, and applying an alternating electric field to the target site of the subject, wherein the target site is at the site of the spinal cord injury.

In some aspects, the composition further comprises neural stem progenitor cells; the biomaterial scaffold comprises neural stem progenitor cells; or the neural stem progenitor cells are administered simultaneously with introducing the composition.

### D. Methods of Increasing N-cadherin

Disclosed herein are methods of increasing N-cadherin expression in a subject having spinal cord injury comprising applying alternating electric fields, at a frequency for a period of time, to a target site of the subject, thereby increasing N-cadherin expression at the target site in the subject. In some aspects, the target site is the site of the spinal cord injury.

In some aspects, N-cadherin regulates mechanical adhesion between neural stem/progenitor cells (NSPCs), and also drives NSPCs migration and promotes NSPCs differentiation.

In some aspects, the subject having a spinal cord injury does not have cancer, has not been diagnosed with cancer, and/or is not being treated for cancer. Thus, in some aspects, alternating electric fields are applied to a subject that does not have cancer and/or has not yet been diagnosed with cancer. In some aspects, the target site does not comprise cancer cells and/or a tumor(s).

In some aspects, the methods disclosed herein can further comprise the step of implanting a collagen scaffold to the site of the spinal cord injury before or after applying an alternating electric field, at a frequency for a period of time to the subject. In some aspects, the collagen scaffold is a linear ordered collagen scaffold. In some aspects, the collagen scaffold can promote the migration and neuronal differentiation of neural stem/progenitor cells to the spinal cord injury to aide in repair. Thus, the application of a collagen scaffold with the alternating electric fields can increase N-cadherin and can be an effective treatment for a spinal cord injury, can increase N-cadherin expression at the target site, can increase axon regeneration, can increase neuronal protection or neuronal differentiation at the target site, and/or can increase neuronal connections at the target site or at or around the collagen scaffold. Because a collagen scaffold can be implanted at the site of a spinal cord injury, in some aspects, the target site for applying alternating electric fields can comprise an implanted collagen scaffold. In some aspects, the disclosed methods can further comprise applying an alternating electric field, at a frequency for a period of time to the site of the spinal cord injury after implanting a linear ordered collagen scaffold to the site of the spinal cord injury.

In some aspects, the methods can further comprise the step of introducing a biomaterial scaffold to the site of the spinal cord injury before or after applying an alternating electric field, at a frequency for a period of time to the subject. In some aspects, introducing the biomaterial scaffold comprising implanting the biomaterial scaffold. In some aspects, biomaterial scaffolds can include, but are not limited to, hydrogels, collagen scaffolds, biodegradable scaffolds, micro/nanofibers as instructive biomaterials, and drug-delivering biomaterials. For example, a biomaterial scaffold can be one or more of those scaffolds described in Suzuki et al. Current Concepts of Biomaterial Scaffolds and Regenerative Therapy for Spinal Cord Injury. Int. J. Mol. Sci. 2023, 24 (3), 2528, incorporated by reference in its entirety herein.

In some aspects, the alternating electric fields can have any of the frequencies or field strengths described herein. In some aspects, the frequency of the alternating electric field is between 100 kHz and 10 MHz. For example, the frequency of the alternating electric field may be between 50 kHz and 1 MHz or between 100 kHz and 500 kHz. In some aspects, the frequency of the alternating electric field is 100-300 kHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

In some aspects, the methods further comprise ceasing application of the alternating electrical field once spinal cord connectivity is restored.

Disclosed herein are compositions comprising a biomaterial scaffold for use in a method of increasing N-cadherin expression in a subject having spinal cord injury comprising introducing the composition to a target site of a subject; applying an alternating electric field to the target site of the subject, thereby increasing N-cadherin expression at the target site in the subject. In some aspects, the target site is the site of the spinal cord injury.

### E. Methods of Promoting Migration of Neural Stem Progenitor Cells (NSPCs)

Disclosed are methods of promoting migration of neural stem progenitor cells (NSPCs) to a spinal cord injury in a subject comprising applying alternating electric fields, at a frequency for a period of time, to a target site of the subject, wherein the target site is at the site of the spinal cord injury, thereby promoting migration of NSPCs to the target site. In some aspects, NSPCs can replace lost tissue after a spinal cord injury, therefore the migration of NSPCs to a spinal cord injury can be therapeutic.

In some aspects, the target site is the site of the spinal cord injury.

In some aspects, the subject having a spinal cord injury does not have cancer, has not been diagnosed with cancer, and/or is not being treated for cancer. Thus, in some aspects, alternating electric fields are applied to a subject that does not have cancer and/or has not yet been diagnosed with cancer. In some aspects, the target site does not comprise cancer cells and/or a tumor(s).

In some aspects, the methods disclosed herein can further comprise the step of implanting a collagen scaffold to the site of the spinal cord injury before or after applying an alternating electric field, at a frequency for a period of time to the subject. In some aspects, the collagen scaffold is a linear ordered collagen scaffold. In some aspects, the collagen scaffold can promote the migration and neuronal differentiation of neural stem/progenitor cells to the spinal cord injury to aide in repair. Thus, the application of a collagen scaffold with the alternating electric fields can increase N-cadherin and can be an effective treatment for a spinal cord injury, can increase N-cadherin expression at the target site, can increase axon regeneration, can increase neuronal protection or neuronal differentiation at the target site, and/or can increase neuronal connections at the target site or at or around the collagen scaffold. Because a collagen scaffold can be implanted at the site of a spinal cord injury, in some aspects, the target site for applying alternating electric fields can comprise an implanted collagen scaffold. In some aspects, the disclosed methods can further comprise applying an alternating electric field, at a frequency for a period of time to the site of the spinal cord injury after implanting a linear ordered collagen scaffold to the site of the spinal cord injury.

In some aspects, the methods can further comprise the step of introducing a biomaterial scaffold to the site of the spinal cord injury before or after applying an alternating electric field, at a frequency for a period of time to the subject. In some aspects, introducing the biomaterial scaffold comprising implanting the biomaterial scaffold. In some aspects, biomaterial scaffolds can include, but are not limited to, hydrogels, biodegradable scaffolds, micro/nanofibers as instructive biomaterials, and drug-delivering biomaterials. For example, a biomaterial scaffold can be one or more of those scaffolds described in Suzuki et al. Current Concepts of Biomaterial Scaffolds and Regenerative Therapy for Spinal Cord Injury. Int. J. Mol. Sci. 2023, 24 (3), 2528, incorporated by reference in its entirety herein.

In some aspects, the alternating electric fields can have any of the frequencies or field strengths described herein. In some aspects, the frequency of the alternating electric field is between 100 kHz and 10 MHz. For example, the frequency of the alternating electric field may be between 50 kHz and 1 MHz or between 100 kHz and 500 kHz. In some aspects, the frequency of the alternating electric field is 100-300 kHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

In some aspects, the methods further comprise ceasing application of the alternating electrical field once spinal cord connectivity is restored.

Disclosed are compositions comprising a biomaterial scaffold, wherein the biomaterial scaffold comprises neural stem progenitor cells (NSPCs), for use in methods of promoting migration of NSPCs to a spinal cord injury in a subject comprising introducing the composition to a target site of a subject, applying an alternating electric field to the target site of the subject, wherein the target site is at the site of the spinal cord injury, thereby promoting migration of NSPCs to the target site.

### F. Methods of Increasing Neuronal Activities and Characteristics

Disclosed are methods of increasing neuronal activity/function or characteristics by applying alternating electric fields, at a frequency for a period of time, to a target site of the subject, thereby increasing neuronal activity/function at the target site in the subject. Thus, disclosed are the following methods.

Disclosed are methods of increasing neuronal connection in a subject having a spinal cord injury comprising applying alternating electric fields, at a frequency for a period of time, to a target site of the subject, thereby increasing neuronal connection at the target site in the subject. Thus, in some aspects, alternating electric fields allow for increased or better communication between neurons. In some aspects, neuronal connection occurs at the site of the spinal cord injury

Disclosed are methods of increasing axon regeneration in a subject having a spinal cord injury comprising applying alternating electric fields, at a frequency for a period of time, to a target site of the subject, thereby increasing axon regeneration at the target site in the subject. In some aspects, axon regeneration in subjects having a spinal injury is one of the best hopes of returning useful function. In some aspects, axon regeneration can be described as axonal growth from injured neurons. In some aspects, axons can be responsible for transmitting information to neurons and muscles, therefore having axon regeneration after an injury can be critical. In some aspects, axon regeneration occurs at the site of the spinal cord injury.

Disclosed are methods of increasing neuronal protection in a subject having a spinal cord injury comprising applying alternating electric fields, at a frequency for a period of time, to a target site of the subject, thereby increasing neuronal protection at the target site in the subject. Thus, in some aspects, alternating electric fields allow for increased or better protection or neuronal cells by preventing neuronal cell death. In some aspects, neuronal protection occurs at the site of the spinal cord injury

Disclosed are methods of increasing neuronal differentiation in a subject having a spinal cord injury comprising applying alternating electric fields, at a frequency for a period of time, to a target site of the subject, thereby increasing neuronal differentiation at the target site in the subject. In some aspects, neuronal differentiation can promote neuronal regeneration. In some aspects, neuronal differentiation occurs at the site of the spinal cord injury.

In some aspects, the target site is the site of the spinal cord injury.

In some aspects, the subject having a spinal cord injury does not have cancer, has not been diagnosed with cancer, and/or is not being treated for cancer. Thus, in some aspects, alternating electric fields are applied to a subject that does not have cancer and/or has not yet been diagnosed with cancer. In some aspects, the target site does not comprise cancer cells and/or a tumor(s).

In some aspects, the methods disclosed herein can further comprise the step of implanting a collagen scaffold to the site of the spinal cord injury before or after applying an alternating electric field, at a frequency for a period of time to the subject. In some aspects, the collagen scaffold is a linear ordered collagen scaffold. In some aspects, the collagen scaffold can promote the migration and neuronal differentiation of neural stem/progenitor cells to the spinal cord injury to aide in repair. Thus, the combination of a collagen scaffold with the alternating electric fields can increase N-cadherin and can be an effective treatment for a spinal cord injury, can increase N-cadherin expression at the target site, can increase axon regeneration, can increase neuronal protection or neuronal differentiation at the target site, and/or can increase neuronal connections at the target site or at or around the collagen scaffold. Because a collagen scaffold can be implanted at the site of a spinal cord injury, in some aspects, the target site for applying alternating electric fields can comprise an implanted collagen scaffold. In some aspects, the disclosed methods can further comprise applying an alternating electric field, at a frequency for a period of time to the site of the spinal cord injury after implanting a linear ordered collagen scaffold to the site of the spinal cord injury.

In some aspects, the methods can further comprise the step of introducing a biomaterial scaffold to the site of the spinal cord injury before or after applying an alternating electric field, at a frequency for a period of time to the subject. In some aspects, introducing the biomaterial scaffold comprising implanting the biomaterial scaffold. In some aspects, biomaterial scaffolds can include, but are not limited to, hydrogels, collagen scaffolds, biodegradable scaffolds, micro/nanofibers as instructive biomaterials, and drug-delivering biomaterials. For example, a biomaterial scaffold can be one or more of those scaffolds described in Suzuki et al. Current Concepts of Biomaterial Scaffolds and Regenerative Therapy for Spinal Cord Injury. Int. J. Mol. Sci. 2023, 24 (3), 2528, incorporated by reference in its entirety herein.

In some aspects, the alternating electric fields can have any of the frequencies or field strengths described herein. In some aspects, the frequency of the alternating electric field is between 100 kHz and 10 MHz. For example, the frequency of the alternating electric field may be between 50 kHz and 1 MHz or between 100 kHz and 500 kHz. In some aspects, the frequency of the alternating electric field is 100-300 KHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

In some aspects, the methods further comprise ceasing application of the alternating electrical field once spinal cord connectivity is restored.

Disclosed are compositions comprising a biomaterial scaffold for use in methods of increasing neuronal activity/function or characteristics by introducing the composition to a target site of a subject; and applying an alternating electric field to the target site of the subject, wherein the biomaterial scaffold comprises neuronal stem cell progenitor cells or neuronal cells, thereby increasing neuronal activity/function at the target site in the subject.

Disclosed are compositions comprising a biomaterial scaffold for use in methods of increasing neuronal connection in a subject having a spinal cord injury comprising introducing the composition to a target site of a subject; applying alternating electric fields to the target site of the subject, wherein the biomaterial scaffold comprises neuronal stem cell progenitor cells or neuronal cells, thereby increasing neuronal connection at the target site in the subject.

Disclosed are compositions comprising a biomaterial scaffold for methods of increasing axon regeneration in a subject having a spinal cord injury comprising introducing the composition to a target site of a subject; applying alternating electric fields to the target site of the subject, wherein the biomaterial scaffold comprises neuronal stem cell progenitor cells or neuronal cells thereby increasing axon regeneration at the target site in the subject.

Disclosed are compositions comprising a biomaterial scaffold for methods of increasing neuronal protection in a subject having a spinal cord injury comprising introducing the composition to a target site of a subject; applying alternating electric fields to the target site of the subject, wherein the biomaterial scaffold comprises neuronal stem cell progenitor cells or neuronal cells, thereby increasing neuronal protection at the target site in the subject.

Disclosed are compositions comprising a biomaterial scaffold for methods of increasing neuronal differentiation in a subject having a spinal cord injury comprising introducing the composition to a target site of a subject; applying alternating electric fields, at a frequency for a period of time, to a target site of the subject, thereby increasing neuronal differentiation at the target site in the subject. In some aspects, neuronal differentiation can promote neuronal regeneration. In some aspects, neuronal differentiation occurs at the site of the spinal cord injury.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more collagen scaffolds and one or more materials for delivering alternating electric fields, such as the Optune system.

### EMBODIMENTS

Embodiment 1: A method of treating a subject having a spinal cord injury comprising applying an alternating electric field, at a frequency for a period of time, to a target site of the subject, wherein the target site is at the site of the spinal cord injury.

Embodiment 2: The method of embodiment 1, wherein the application of the alternating electric field increases N-cadherin expression at the target site, thereby treating the spinal cord injury in the subject.

Embodiment 3: The method of embodiment 1, wherein the application of the alternating electric field increases axon regeneration, neuronal protection or neuronal differentiation at the target site, thereby treating the spinal cord injury in the subject.

Embodiment 4: The method of embodiment 1, wherein the application of the alternating electric field increases neuronal connections at the target site, thereby treating the spinal cord injury in the subject.

Embodiment 5: A method of increasing N-cadherin expression in a subject having a spinal cord injury comprising applying alternating electric fields, at a frequency for a period of time, to a target site of the subject, thereby increasing N-cadherin expression at the target site in the subject.

Embodiment 6: A method of promoting migration of neural stem progenitor cells (NSPCs) to a spinal cord injury in a subject comprising applying alternating electric fields, at a frequency for a period of time, to a target site of the subject, wherein the target site is at the site of the spinal cord injury, thereby promoting migration of NSPCs to the target site.

Embodiment 7: A method of increasing neuronal connections in a subject having a spinal cord injury comprising applying alternating electric fields, at a frequency for a period of time, to a target site of the subject, thereby increasing neuronal connections at the target site in the subject.

Embodiment 8: A method of increasing axon regeneration in a subject having a spinal cord injury comprising applying alternating electric fields, at a frequency for a period of time, to a target site of the subject, thereby increasing axon regeneration at the target site in the subject.

Embodiment 9: A method of increasing neuronal protection in a subject having a spinal cord injury comprising applying alternating electric fields, at a frequency for a period of time, to a target site of the subject, thereby increasing neuronal protection at the target site in the subject.

Embodiment 10: A method of increasing neuronal differentiation in a subject having a spinal cord injury comprising applying alternating electric fields, at a frequency for a period of time, to a target site of the subject, thereby increasing neuronal differentiation at the target site in the subject.

Embodiment 11: The method of any of the preceding embodiments, wherein the target site is a site of a spinal cord injury.

Embodiment 12: The method of any of the preceding embodiments, wherein the target site comprises a linear ordered collagen scaffold.

Embodiment 13: The method of any of the preceding embodiments, wherein the spinal cord injury occurred at least 1, 2, 3, 4, 5, 6, or 7 days before applying the alternating electric fields.

Embodiment 14: The method of any of the preceding embodiments, further comprising administering physiotherapy, muscle vibration, steroid treatment, respiratory management, and/or pain management.

Embodiment 15: The method of any of the preceding embodiments, wherein the subject does not have cancer, has not been diagnosed with cancer, or is not being treated for cancer.

Embodiment 16: The method of any of the preceding embodiments, wherein the frequency of the alternating electric field is between 50 kHz and 1 MHZ.

Embodiment 17: The method of any of the preceding embodiments, wherein the frequency of the alternating electric field is 210-400 kHz.

Embodiment 18: The method of any of the preceding embodiments, wherein the alternating electric field has a field strength of between 0.5 and 4 V/cm RMS.

Embodiment 19: The method of any of the preceding embodiments, wherein the alternating electric field has a field strength of 0.9 V/cm RMS.

Embodiment 20: The method of any of the preceding embodiments, wherein axon regeneration at the site of the spinal cord injury is increased compared to axon regeneration at a spinal cord injury site in the absence of alternating electric fields.

Embodiment 21: The method of any of the preceding embodiments, wherein neuronal differentiation at the site of the spinal cord injury is increased compared to neuronal differentiation at a spinal cord injury site in the absence of alternating electric fields.

Embodiment 22: The method of any of the preceding embodiments, further comprising ceasing application of the alternating electrical field once spinal cord connectivity is restored.

Embodiment 23: The method of any of the preceding embodiments, further comprising implanting a linear ordered collagen scaffold to the site of the spinal cord injury.

Embodiment 24: The method of embodiment 23, further comprising applying an alternating electric field, at a frequency for a period of time to the site of the spinal cord injury after implanting a linear ordered collagen scaffold to the site of the spinal cord injury.

Embodiment 25: A composition comprising a biomaterial scaffold for use in a method of treating a subject having a spinal cord injury comprising introducing the composition to a target site of a subject, and applying an alternating electric field to the target site of the subject, wherein the target site is at the site of the spinal cord injury.

Embodiment 26: The composition of embodiment 25, wherein the composition further comprises neural stem progenitor cells; wherein the biomaterial scaffold comprises neural stem progenitor cells; or wherein neural stem progenitor cells are administered simultaneously with introducing the composition.

Embodiment 27: The composition of embodiment 26, wherein the biomaterial scaffold is a linear ordered collagen scaffold, hydrogel, biodegradable scaffold, and/or micro/nanofiber scaffold.

Embodiment 28: The composition of embodiment 25-27, wherein the application of the alternating electric field increases N-cadherin expression at the target site, thereby treating the spinal cord injury in the subject, and/or wherein the application of the alternating electric field increases axon regeneration, neuronal protection or neuronal differentiation at the target site, thereby treating the spinal cord injury in the subject, and/or wherein the application of the alternating electric field increases neuronal connections at the target site, thereby treating the spinal cord injury in the subject.

Embodiment 29: The composition of any of the preceding embodiments, wherein the target site is a site of a spinal cord injury.

Embodiment 30: The composition of any of the preceding embodiments, wherein the spinal cord injury occurred at least 1, 2, 3, 4, 5, 6, 7, 8, 9, 10, 11, 12, 13, 14, 15, 16, 17, 18, 19, 20, 21, 22, 23, or 24 hours before applying the alternating electric fields.

Embodiment 31: The composition of any of the preceding embodiments, further comprising administering physiotherapy, muscle vibration, steroid treatment, respiratory management, and/or pain management to the subject.

Embodiment 32: The composition of any of the preceding embodiments, wherein the subject does not have cancer, has not been diagnosed with cancer, or is not being treated for cancer.

Embodiment 33: The composition of any of the preceding embodiments, wherein the alternating electric field is applied at a frequency between 50 kHz and 1 MHz, preferably 210-400 kHz.

Embodiment 34: The composition of any of the preceding embodiments, wherein the alternating electric field has a field strength of between 0.5 and 4 V/cm RMS, preferably 0.9 V/cm RMS.

Embodiment 35: The composition of any of the preceding embodiments, wherein axon regeneration at the site of the spinal cord injury is increased compared to axon regeneration at a spinal cord injury site in the absence of alternating electric fields, and/or wherein neuronal differentiation at the site of the spinal cord injury is increased compared to neuronal differentiation at a spinal cord injury site in the absence of alternating electric fields.

Embodiment 36: The composition of any of the preceding embodiments, further comprising ceasing application of the alternating electrical field once spinal cord connectivity is restored.

Embodiment 37: A kit comprising a biomaterial scaffold and one or more materials for delivering an alternating electric field.

### EXAMPLES

N-cadherin (N-cad) modification improved the adhesion of endogenous neural stem/progenitor cells on collagen scaffold and increased the differentiation into neurons. When a linear ordered collagen scaffold (LOCS)-Ncad was transplanted into complete transected rat spinal cords, more NSPCs migrated to the lesion center and more newborn neurons appeared within the injury site. Furthermore, rats transplanted with LOCS-Ncad showed significantly improved locomotor recovery compared with the rats without implants.

Deleting Ncad in astrocytes decreases PTEN-knock-down-induced RGC axon regeneration.

N-cadherin engagement, in the absence of other survival factors (cell-matrix interactions and serum), protects GT1-7 neuronal cells against apoptosis.

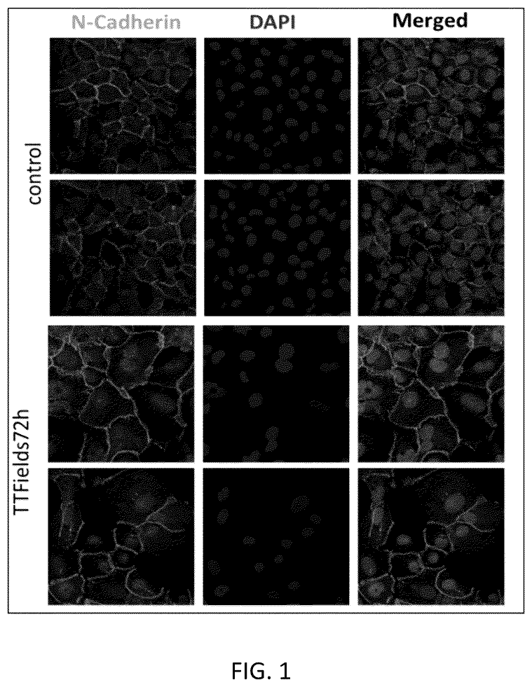

Current experiments show that N-cadherin expression is increased in ECM secreting cells (fibroblasts) following TTFields application (FIG. 1).

Those skilled in the art will recognize, or be able to ascertain using no more than routine experimentation, many equivalents to the specific embodiments of the methods and compositions described herein. Such equivalents are intended to be encompassed by the following claims.

### REFERENCES

Prakasam et al., Similarities between heterophilic and homophilic cadherin adhesion, PNAS, 2006 Oct. 17; 103(42):15434-15439
Zhang et al., Cadherin-based biomaterials: Inducing stem cell fate towards tissue construction and therapeutics, Progress in Natural Science: Materials International, vol 30(5), October 2020, pages 597-608
Miyamoto et al., N-cadherin-based adherens junction regulates the maintenance, proliferation, and differentiation of neural progenitor cells during development, Cell Adhesion & Migration, vol 9(3), 2015, pages 183-192
Liu et al., A functional scaffold to promote the migration and neuronal differentiation of neural stem/progenitor cells for spinal cord injury repair, Biomaterials, 2020 June; 243:119941 Ribeiro et al., Neural Cadherin Plays Distinct Roles for Neuronal Survival and Axon Growth under Different Regenerative Conditions, ENeuro, September 2020, 7(6)
Lelievre et al., N-cadherin mediates neuronal cell survival through Bim down-regulation, PLoS One, 2012;7(3):e33206

We claim:

1. A method of treating a subject having a spinal cord injury comprising:
applying an alternating electric field, at a frequency for a period of time, to a target site of the subject,
wherein the target site is at the site of the spinal cord injury.

2. The method of claim 1, wherein the application of the alternating electric field increases N-cadherin expression at the target site, thereby treating the spinal cord injury in the subject.

3. The method of claim 1, wherein the application of the alternating electric field increases axon regeneration, neuronal protection or neuronal differentiation at the target site, thereby treating the spinal cord injury in the subject.

4. The method of claim 1, wherein the application of the alternating electric field increases neuronal connections at the target site, thereby treating the spinal cord injury in the subject.

5. A method of increasing N-cadherin expression in a subject having a spinal cord injury comprising:
applying alternating electric fields, at a frequency for a period of time, to a target site of the subject,
thereby increasing N-cadherin expression at the target site in the subject.

6. A method of increasing axon regeneration in a subject having a spinal cord injury comprising:
applying alternating electric fields, at a frequency for a period of time, to a target site of the subject,
thereby increasing axon regeneration at the target site in the subject.

7. The method of claim 1, wherein the target site is a site of a spinal cord injury.

8. The method of claim 1, wherein the target site comprises a linear ordered collagen scaffold.

9. The method of claim 1, wherein the spinal cord injury occurred at least 1, 2, 3, 4, 5, 6, or 7 days before applying the alternating electric fields.

10. The method of claim 1, further comprising administering physiotherapy, muscle vibration, steroid treatment, respiratory management, and/or pain management.

11. The method of claim 1, wherein the subject does not have cancer, has not been diagnosed with cancer, or is not being treated for cancer.

12. The method of claim 1, wherein the frequency of the alternating electric field is between 50 kHz and 1 MHz.

13. The method of claim 1, wherein the frequency of the alternating electric field is 210-400 kHz.

14. The method of claim 1, wherein the alternating electric field has a field strength of between 0.5 and 4 V/cm RMS.

15. The method of claim 1, wherein the alternating electric field has a field strength of 0.9 V/cm RMS.

16. The method of claim 1, wherein axon regeneration at the site of the spinal cord injury is increased compared to axon regeneration at a spinal cord injury site in the absence of alternating electric fields.

17. The method of claim 1, wherein neuronal differentiation at the site of the spinal cord injury is increased compared to neuronal differentiation at a spinal cord injury site in the absence of alternating electric fields.

18. The method of claim 1, further comprising ceasing application of the alternating electrical field once spinal cord connectivity is restored.

19. The method of claim 1, further comprising implanting a linear ordered collagen scaffold to the site of the spinal cord injury.

20. The method of claim 19, further comprising applying an alternating electric field, at a frequency for a period of time to the site of the spinal cord injury after implanting a linear ordered collagen scaffold to the site of the spinal cord injury.
